(19) United States
(12) Patent Application Publication
(10) Pub. No.: US 2005/0239107 A1
(43) Pub. Date: Oct. 27, 2005

(54) Trapped photochromes and thermochromes and uses for chromogenic detection, including detection of airborne contaminants

(76) Inventor: J. Thomas Ippoliti, Woodbury, MN (US)

Correspondence Address: PAUL PARINS;Kagan Binder PLLC, 221 Main St N Ste 200, 200 Maple Island Building, Stillwater, MN, 55082, US

(21) Appl. No.: 11/051,097

(22) Filed: Feb. 3, 2005

Related U.S. Application Data
(60) Provisional application No. 60/541,351, filed on Feb. 3, 2004.
(60) Provisional application No. 60/541,350, filed on Feb. 3, 2004.

(51) Int. Cl. C12Q 1/68; G01N 33/543; C07D 471/10

(52) U.S. Cl. 435/6; 436/518; 546/17; 530/409

(57) ABSTRACT

The present invention relates to methods of making a trapped chromogenic compound (including photochromic and thermochromic compounds) by causing an open form of the chromogenic compound and a compound comprising an electrophilic moiety to be covalently bonded. In a preferred embodiment, the trapped, open, chromogenic compound includes an ester moiety derived from ingredients including an oxygen atom of an open pyran moiety and an acyl functional compound. The present invention also relates to trapped, open, chromogenic compounds and methods of using them. Preferred methods of using such compounds include methods of detecting biological molecules and/or toxic chemicals.

### PRIORITY CLAIM

The present non-provisional patent Application claims priority under 35 USC §119(e) from U.S. Provisional Patent Applications having Ser. No. 60/541,351, filed on Feb. 3, 2004, by Ippoliti and titled TRAPPED PHOTOCHROMES AND THERMOCHROMES AND USES FOR CHROMOGENIC DETECTION, and Ser. No. 60/541,350, filed Feb. 3, 2004, by Ippoliti and titled TRAPPED PHOTOCHROMES AND THERMOCHROMES AND USES FOR CHROMOGENIC DETECTION, INCLUDING DETECTION OF AIRBORNE CONTAMINANTS, wherein the entireties of said provisional patent applications are incorporated herein by reference.

### FIELD OF THE INVENTION

The present invention relates to chromogenic molecules of the type having open and closed configurations, methods of trapping the molecules in the open configuration, the resultant trapped molecules, and methods of using such trapped molecules for diagnostic or detection purposes. More particularly, the present invention relates to photochromic compounds that are reversibly ‘trapped’ in their open, typically colored state, via covalent bonding to an electrophile. The inventive trapped, colored photochromic compounds are advantageously stable. For instance, a photochromic molecule containing a ring-opening pyran moiety with a nucleophilic O atom can be trapped open using an acyl halide functionalized enzyme substrate via a reaction that causes the enzyme substrate to be covalently linked to the O atom via an ester linkage. The resultant trapped molecule can then be used to diagnostically detect the presence of the corresponding enzyme. In the presence of the enzyme, the enzyme substrate is cleaved from the chromogenic molecule, which generally allows the chromogenic molecule, now in its open form, to return to its closed configuration and thereby change color. The change of color thus indicates the presence of the enzyme.

### BACKGROUND OF THE INVENTION

Photochromic and Thermochromic Compounds:

A chromogenic compound, also referred to as a chromogen, refers to a compound that changes color when it undergoes a chemical reaction. Compounds that exhibit a color change in the presence of thermal energy are generally known thermochromic compounds, while those that change color upon exposure to ultraviolet or other electromagnetic energy are known as photochromic compounds. Some compounds are both photochromic and thermochromic. The compounds generally revert back to their original state of color (which are normally colorless) when the exciting energy is removed. Many photochromic compounds are known and are described, for instance, in U.S. Pat. Nos. 6,211,374; 6,478,988; 5,543,533; 5,532,361; 4,882,438; 4,220,708; 3,501,410; and 3,100,778, the entireties of which are incorporated herein by reference. In many photochromic compounds, the exciting energy has been shown to cause a change in the compound from a first molecular configuration to a second configuration. More particularly, photochromic compounds can form an open form with an extended conjugated system capable of absorbing visible light and are therefore colored. When the source of electromagnetic radiation is removed, the compounds can revert to their original structure (closed form) which is colorless. As the open configuration of these compounds is typically very unstable relative to the closed configuration, this reversion typically occurs very quickly. One class of such photochromic compounds are pyran-based in the sense that these compounds incorporate a pyran ring moiety. Color changes in such compounds result from configurational changes in the pyran moiety. In one form most often corresponding to the normal or ground state, the pyran ring is closed. In another form corresponding to a higher energy state, exposure to light causes the pyran ring to open. The open form has a more extended, conjugated system of electrons than the closed form. Consequently, these open molecules absorb visible light differently and are therefore colored differently than the closed form. When the light is removed, the molecules tend to revert to their closed structure, which often is colorless. The rate at which the open form reverts back to the closed form varies from molecule to molecule, but is relatively short in most instances, e.g., from less than a second to minutes. The rate at which such reverting occurs generally corresponds to the stability of the open form. With greater stability, the open form takes longer to revert back to the closed form. With lower stability, reverting occurs more rapidly.

The closed and open forms of a representative indolinospiropyran are shown below.

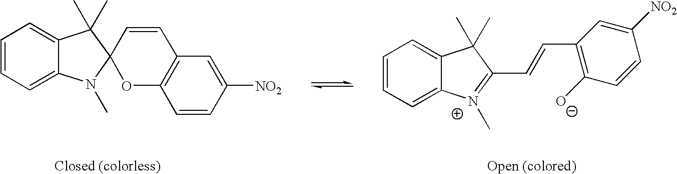

Another example of a similar compound is the molecule 1,3,3 trimethylindolino-p-napthopyrylospiran that is excited by UV light (or heat) and undergoes the structural rearrangement into an open conjugated form. This open form is highly polarized, as illustrated by the zwitterionic resonance structure shown.

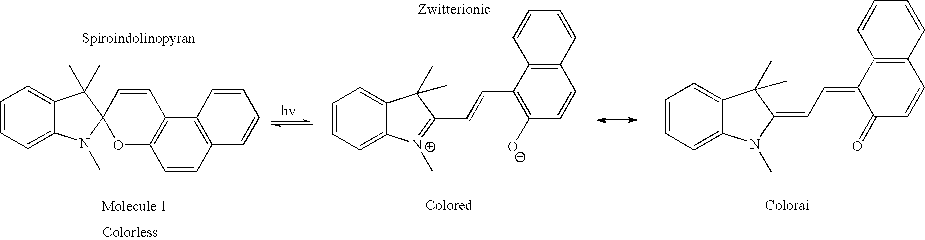

Many other classes of other photochromic compounds that have open and closed ring forms are also known and can be found in the following general references, each of which is incorporated herein by reference in its entirety: Peter Bamfield, “Chromic Phenomena: Technological Applications of Colour Chemistry”, The Royal Science of Chemistry, Cambridge, UK, 2001; “Infrared Absorbing Dyes”, Masaru Matsuoka, ed., Plenum Press, New York, 1990; “Organic Photochromic and Thermochromic Compounds, Volume 1: Main Photochromic Families”, John C. Crano and Robert J. Guglilmetti eds., Plenum Press, New York, N.Y., 1999; “Organic Photochromic and Thermochromic Compounds, Volume 2: Physiochemical Studies, Biological Applications, and Thermochromism”, John C. Crano and Robert J. Guglilmetti eds., Kluwer Academic/Plenum Publishers, New York, N.Y., 1999; and “Biological Applications of Photochemical Switches”, Harry Morrison, ed., John Wiley & Sons, Inc., New York, N.Y., 1993.

Photochromic molecules are used in a wide variety of applications, including uses as reversible photoactive dyes in novelty items (such as nail polish and t-shirts), or in photosensitive plastic ophthalmic lenses.

Although these compounds have been successfully employed in commercial applications where their quickly reversing nature is advantageously exploited or tolerable, as the case may be, such as in photosensitive ophthalmic lenses, they could find additional application if the colored forms thereof were more stable. Perhaps in recognition of this unmet potential, numerous attempts have been made to provide photochromic compounds stable, or ‘trapped’ in the open configuration. As a result, a small number of trapped photochromic compounds have been prepared, typically relying upon the interaction of the photochromic compound with a metallic or organometallic compound or a proton in order to trap the photochrome in its open colored state. While advantageous in certain applications, these trapped photochromes can yet be less stable than desirable for certain applications, and further, are typically not soluble in organic solvents, further limiting their utility.

Photochromic compounds have been trapped in their open form by metal ions as described in Wojtyk et al., “Modulation of the Spiropyran-Merocyanine Reversion via Metal-Ion Selective complexation: Trapping of the “Transient” cis-Merocyanine,” Chem. Mater. 2001, 13 2547-2551 (2001). There are also examples of trapping the open form of a photochromic compound with acids to give the protonated open form.

It would be desirable and advantageous in many applications to provide a photochromic compound that could be trapped in its open configuration and yet have sufficient stability to be commercially feasible in applications where at least a limited shelf life may be desired or required. It would further be desirable to provide stable, trapped open, photochromic compounds that were also soluble in organic solvents so as to be useful in applications where a liquid formulation is desirable.

### SUMMARY OF THE INVENTION

The present invention relates to improved photochromic compounds, as well as methods of using these molecules for a variety of purposes, including to detect, e.g., enzyme activity or the presence of toxic chemicals such as nerve agents. More particularly, the present invention relates to photochromic compounds that are reversibly ‘trapped’ in their open, colored state, via covalent bonding to an electrophile. The inventive trapped, colored photochromic compounds are advantageously stable and may be further soluble in organic solvents in some embodiments.

According to one aspect of the present invention, a method of making a trapped chromogenic compound includes the step of causing an open form of a chromogenic compound and a compound including an electrophilic moiety to be covalently bonded.

According to another aspect of the present invention, a photochromic compound trapped in an open configuration has the following structure:
Z-W-R
wherein Z is derived from ingredients comprising any photochomic compound including a pyran moiety and Z is present in a configuration in which the pyran moiety is open, W is a single bond or a divalent linking group linked to the open pyran moiety, and R is at least a portion of the residue of a compound comprising an electrophilic moiety.

According to another aspect of the present invention, a method for detecting the presence of a toxic chemical in a sample includes contacting the sample with a photochromic compound capable of being trapped into an open configuration via covalent bonding with the toxic chemical and visually observing the photochromic compound for a change in color.

According to another aspect of the present invention, a method of providing a detection molecule includes the steps of determining information indicative of the rate at which an untrapped, open chromophore reverts to a closed configuration; and using the information to make a trapped open chromophore having a desired colorimetric rate of response in a detecting application.

### BRIEF DESCRIPTION OF DRAWINGS

FIG. 1 shows an NMR obtained for the molecule after trapping molecule 1 with 2 Benzenesulfonylamino-propionyl chloride.

FIG. 2 shows an NMR of DCP reacted with the thermochromic indicator.

FIG. 3 shows an NMR of HCl bubbled through the thermochromic indicator.

FIG. 4 shows an NMR of DCP reacted with the thermochromic indicator in the glove box.

FIG. 5 shows a second NMR of DCP reacted with the thermochromic indicator in the glove box (one week later).

FIG. 6 shows an absorption scan of the thermochromic indicator in diethyl ether.

FIG. 7 shows an absorption scan of the thermochromic indicator in chloroform.

FIG. 8 shows kinetics of color change.

### DETAILED DESCRIPTION

In some aspects, the present invention describes the formation of dyes by covalently trapping the open form of photochromes or thermochromes with an electrophilic reagent. Although the open form is present in very low concentrations trapping to form an insoluble salt in ether drives the reaction to completion. More specifically, in such aspects the open form of an indolinospiropyran was trapped out with an acid chloride to form a highly colored ester. (Example 1)

In the present invention, photochromic molecules, especially those based upon pyran, are trapped in an open configuration by covalently reacting the open configuration of such molecules with a reactive reagent, desirably an electrophilic reagent. In preferred embodiments, this is accomplished by reacting the O atom of an open pyran ring with an electrophilic compound, such as an acyl halide, in which the O atom is thereby incorporated into an ester linkage. With the O atom thus trapped, the compound tends to remain in the open configuration and display a color that is distinct from that of the ground state color (if any). This trapped form is now a chromogen, a compound that can change color by undergoing a chemical reaction. The trapped compounds tend to exhibit relatively stable color characteristics until the ester linkage is cleaved resulting in the O atom becoming untrapped. The stability of the trapped open form generally is not related to the rate at which the untrapped form cyclizes back to the closed form. The ester linkage is readily cleaved under a variety of reaction conditions, to free the O atom and allow the molecule to revert with an accompanying color change to its ground state the closed form.

In a representative reaction scheme, a photochrome incorporating a pyran moiety and a compound containing an acyl halide are reacted together to from the trapped chromogen. The reaction preferably occurs with heat and/or in the presence of ultraviolet light so that the photochrome is in the open form. The acyl halide moiety and the open O of the pyran moiety covalently react to form an ester linkage.

The reaction theoretically involves a 1:1 stoichiometry between acyl halide and the open O atom. However, to help ensure that the reaction goes to completion, it is desirable in some circumstances to use a slight excess of the acyl halide.

A variety of different kinds of pyran-based chromogens would be suitable in the practice of the invention and include any found in “Organic Photochromic and Thermochromic Compounds, Volume 1: Main Photochromic Families”, John C. Crano and Robert J. Guglilmetti eds., Plenum Press, New York, N.Y., 1999.

In some modes of practice, it may be desirable to trap the photochrome open with a compound that, as supplied, may not incorporate an acyl halide moiety. For instance, any carboxylic acid can be converted to an acid chloride and then function as an electrophilic trapping reagent. Also a phosphoryl chloride can function as an electrophilic trapping agent. The resultant phosphate ester could then function as an enzyme substrate.

The reaction generally proceeds better in an organic solvent in which the water content is restricted to at least 10% by weight or less, preferably about 5% by weight or less, and more preferably about 0.5% by weight or less of water based upon the total weight of the water and other solvent(s). Examples of suitable organic solvents include diethyl ether and methylene chloride, combinations of these, and the like. A particularly preferred solvent is an anhydrous ether.

The trapped chromogen tends to remain in the trapped state until the covalent linkage between the pyran O atom and the trapping moiety is cleaved to release the O atom. The linkage is fairly stable in the ambient, even if the ambient is humid, and the trapped chromogen may be stored in a suitable container for relatively long periods of time without undue loss of color. However, cleaving to untrap the chromogen may occur or be caused to occur in a variety of ways. One basic mode of cleavage is hydrolysis. The hydrolysis reaction can be catalyzed by strong acid or base, but preferably by an enzyme.

The conditions under which the trapped form is released and allowed to revert to the ground state may be controlled so that the reverting occurs only when certain conditions are present. This allows the trapped compound to be incorporated into a colorimetric detector for such conditions. Thus, in one aspect of the invention, the principles of the invention may be used to calorimetrically detect the presence of one or more biological molecules such as an enzyme, protein, RNA, DNA, sugar, etc. Generally, one or more trapping compounds co-reactive with the biological molecule of interest are used to trap one or more photochromic compounds in an open configuration. The trapped, open, chromogenic reaction product is then exposed to the sample of interest. If the biological molecule of interest is present, the biological molecule will tend to react with the trapped chromogen, cleaving it from the open form of the photochrome that then rapidly closes. The resultant color change of the chromogen as it reverts back to its ground state thus colorimetrically signals detection of the biological molecule of interest. A lack of color change would indicate that the molecule is not present.

For example, consider a circumstance in which it may be desirable to detect the presence of leukocytes. This kind of detection may be desirable inasmuch as leukocytes may be indicative of illness or disease. Compounds for detecting the presence of leukocytes, in particular by using esters that are cleaved by enzymatic activity resulting in the formation of color are well known and can be found in the following patents: U.S. Pat. Nos. 4,299,917 and 4,704,460 and British Patent No. 1,128,371. The detection strategy may involve either detecting the leukocyte directly or indirectly. As an example of an indirect detection technique, it is not the leukocyte that is detected directly but rather leukocyte esterase, which is an enzyme that typically is present in mucus, urine or other bodily fluid or tissue samples when leukocytes are present. Thus, for indirect detection, the photochrome is trapped open with a compound that is reactive with leukocyte esterase. Such a compound, sometimes referred to as an enzyme substrate, may be modified if needed to incorporate acyl halide functionality so as to be reactive with the open photochrome. In the indirect strategy, it is really the enzyme that is being detected directly, from which the presence of the leukocyte is then inferred.

Whether a direct or indirect detection strategy is used, the trapped molecule may then be used to detect the presence of the leukocyte in the sample of interest. The trapped molecule may be used per se or optionally may be incorporated into a detection medium or device such as a test strip, diaper or other undergarment, ear swab, towel, sanitary napkin, testing cell, or the like. The sample of interest is caused or allowed to contact the trapped molecule, which now functions as a calorimetric detector for leukocytes (either via direct or indirect detection). If the leukocyte (or leukocyte esterase) is present, a color change indicates that the trapped chromogen and the biological molecule to be detected have reacted, untrapping the chromogen and allowing the photochrome to revert to the closed configuration and color of its ground state. Thus, a color change is a positive indicator for the presence for the leukocyte or leukocyte esterase enzyme, as the case may be.

The following reaction scheme shows schematically one illustrative way of making a trapped chromogen that would be able to detect the presence of leukocyte esterase, and hence leukocytes. An acyl halide functional embodiment of a leukocyte esterase substrate, i.e., a compound that is co-reactive with leukocyte esterase enzyme, is reacted with a photochromic indolinospiropyran. These are reacted together in dry MeCl₂ in the presence of UV light. The acyl halide forms an ester linkage with the O of the open pyran ring, trapping the photochromic compound in its open configuration. The resultant detection molecule has a deep red-orange color. In the presence of leukocyte esterase, the trapping moiety is cleaved from the detection molecule. This untraps the O atom of the pyran and allows the ring to close. The closed form of the photochrome is generally colorless. The change of color from orange to colorless thus provides a positive detection signal for leukocyte esterase.

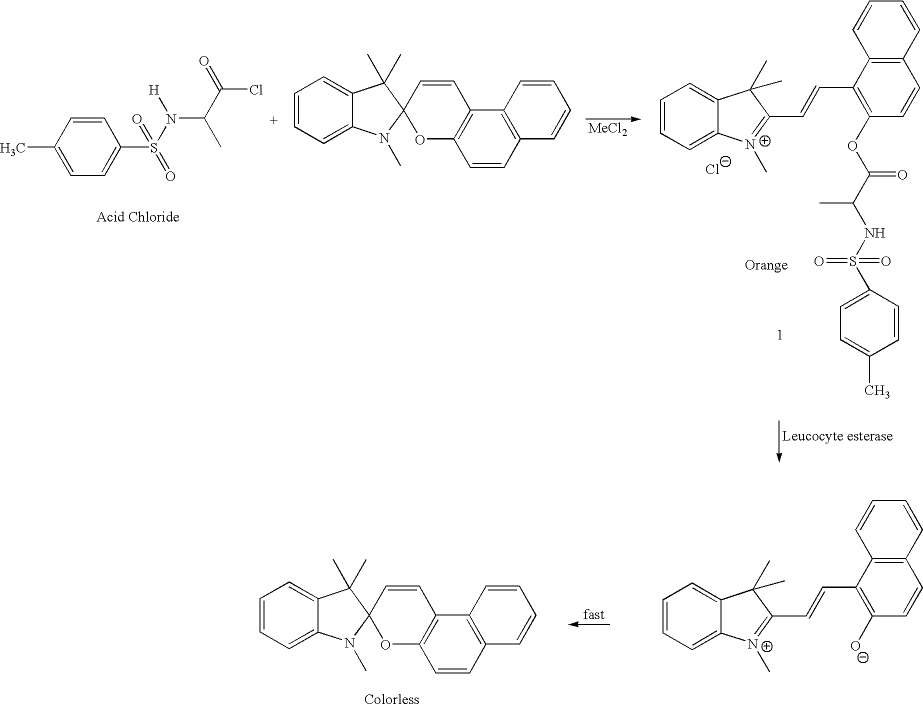

Other representative embodiments of the invention include utilization of photochromic and thermochromic molecules for detecting other kinds of compounds or conditions. As just one illustrative example, photochromic or thermochromic molecules could be used to calorimetrically detect substances in the ambient air. For example, entities such as police or military forces may need to test the ambient air for nerve agents such as volatile organophosphorus compounds that react with hydroxyl groups in biological tissue to form a phosphate ester. When this reaction occurs at the site of acetylcholinesterase—an enzyme critical to nerve function—the enzyme's activity could be inhibited, leading potentially to convulsions, serious injury, or even death. Examples of such nerve agents include sarin and diisopropylfluorophosphate. Examples of utilizing a compound in this manner is shown in the scheme below for a thermochromic molecule.

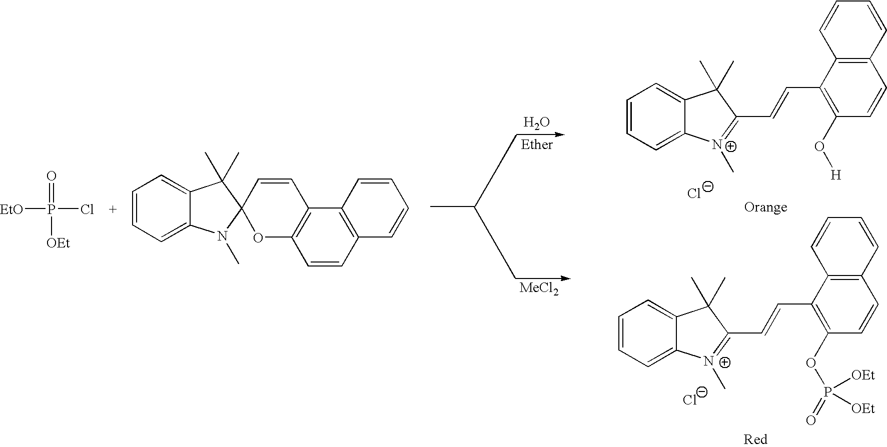

In the presence of a nerve agent, the nerve agent would react with the open form of the thermochrome, which is present even at room temperature to some degree, to form a highly colored phosphate ester, thus detecting the presence of the nerve agent. We have shown that this compound reacts rapidly with less reactive phosphoryl chlorides (like diethyl chlorophosphate shown in the scheme directly above), in the presence of water, to give the highly colored protonated open form in ether, acting again as a detector for such compounds. The molecule also reacts, when water is not present, directly with the open form of the thermochrome to form a phosphate ester that is red.

Rate of Color Change

The nature of the untrapped chromogen can impact the rate at which a color change occurs when the trapping moiety on a trapped chromogen is cleaved to allow the open chromogen to revert to its closed configuration. Thus, if one wants to provide a colorimetric sensor molecule that delivers a very rapid color change to signal positive detection, the detection molecule may incorporate a chromogen whose open configuration, when untrapped, is relatively less stable and, therefore, tends to revert more rapidly to the closed configuration than relatively more stable chromogens. Conversely, if one wants the colorimetric sensor to deliver a color change less rapidly, the detection molecule may incorporate a chromogen whose untrapped, open configuration is relatively stable.

Other embodiments of trapped chromogens of the invention also have been synthesized and are shown below as compounds 2-4.

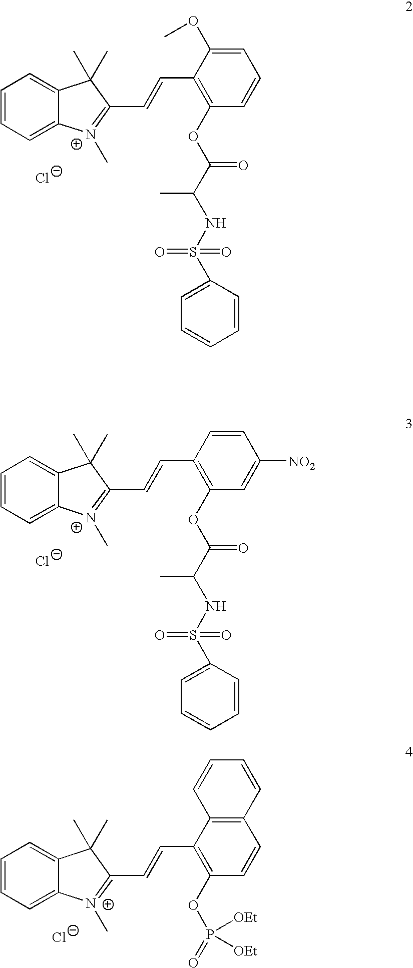

One class of trapping compounds described above include acyl halide functionality in which the halide may be Cl, F, or the like. However, other anionic species may also be used as a counterion. For instance, some embodiments of the invention have some degree of apparent instability in water and ethanol solutions as indicated by change in color. Even in DMF, the solutions turn yellow, although yellow is not the color of the cleaved form. While not wishing to be bound by theory, it is believed that such color and potentially configurational instability may result from because the solvent, e.g. water, is adding to the carbon adjacent to the nitrogen as shown below. This disrupts some of the conjugation, thus changing the color to yellow. One reason that this might occur is that there is a lot of charge localized on the carbon. There are at least 2 possible ways to alleviate this problem and also stabilize the trapped form. One possible solution would be to change the counter-ion of the trapping compound from a halide (e.g., chloride) to tetrafluoroborate (BF₄⁻).

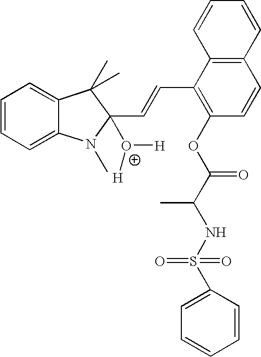

Another way is to use photochromic molecules where the charge is spread out more such as compound 6 below, where C designates the closed form and O designates the open form. The open form (6O) would be readily trapped out using the methodology of the present invention. Photochrome 6 is a known photochrome.

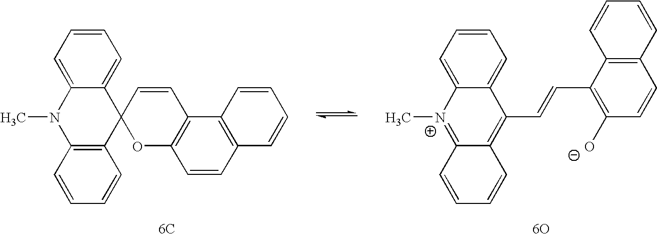

To spread the charge out even more, the following novel photochromic molecule could be synthesized.

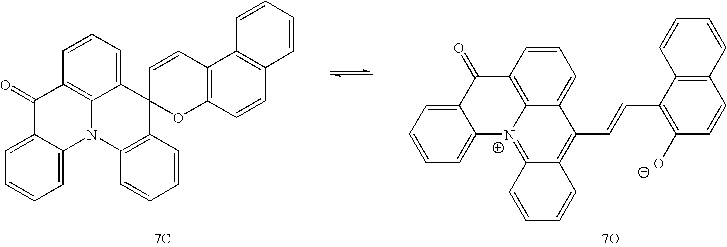

The carbonyl moiety on the compound 7C/7O provides a facile way to attach the compound to a substrate such as a polymer or ELISA conjugate to thereby incorporate color changing properties into the substrate. The photochromic molecule can be made from the known compound 7-SM by the route shown below.

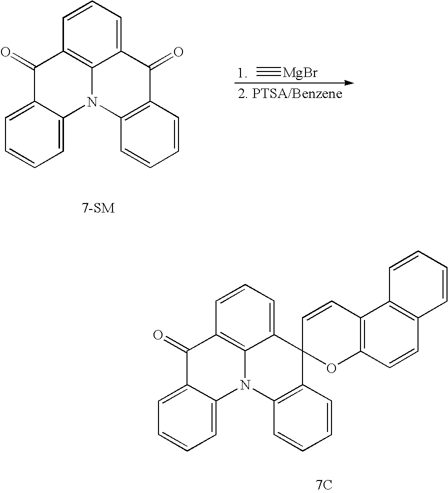

Also, the trapped form of the starting material used to make 7 may be colored, and it certainly should be fluorescent so it may have some value as a detection molecule also such as in the following mode of practice in which the trapping molecule is a leukocyte esterase substrate:

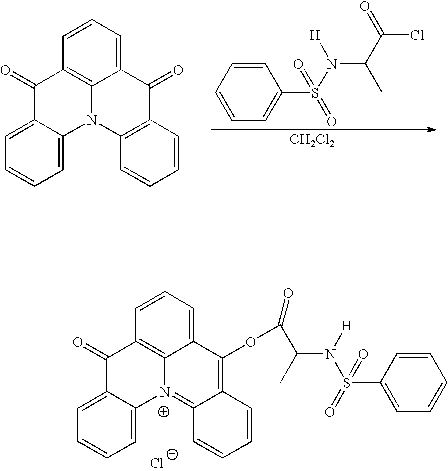

Uniquely, a naphthospiropyran also has been trapped out with a proton as described in below. In fact, using HBF₄ they are very stable in air (e.g. see below, this salt is a deep blue). These may be useful in detection of other compounds or conditions, including counterfeit detection. 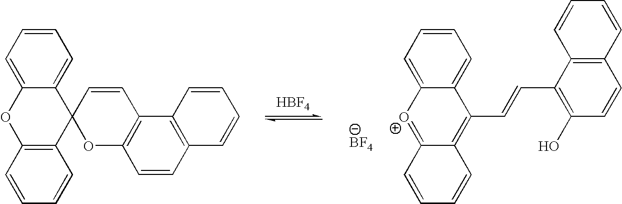
Representative Examples Illustrating Exemplary Practices of the Present Invention Synthesis of RF1P35A Photochrome:

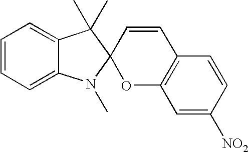

A 500 mL two neck Round Bottom flask equipped with a stir bar was flame dried and flushed with Nitrogen and attached to a reflux condenser. 1,2,3,3-tetramethyl-3H-indolium iodide (2.0 g, 6.64 mmols) was added to the flask along with 90 mL of ethanol. The solution was stirred and left under Nitrogen. The solid did not dissolve. Triethylamine (1.39 mL, 1.0 g, 9.96 mmols) was added to the reaction via a disposable syringe and needle. 2-hydroxy-5-nitro benzaldehyde (1.12 g, 6.64 mmols) was added to the reaction and the solution was heated and refluxed under positive Nitrogen pressure for three hours. A dark purple color change was observed. The reaction mixture was left to cool to room temperature followed by a brief 20 minute cooling with an ice bath. The yellow precipitate product was collected by vacuum filtration and washed with ethanol. A TLC was taken of the product in an ethanol solution using a 95:5 ratio of hexanes/ethyl acetate for a developing solvent. The TLC showed a pure product. The product was weighed as 1.49 g having a calculated theoretical yield of 2.14 g and a percent yield of 69.6%.

Examples Involving Napthospiropyran Photochromes:

The following examples describe experiments to trap out napthospiropyran photochromes in their open forms with acid chlorides. The experimental approach was to take advantage of the nucleophilic properties of pyran oxygens by subjecting napthospiropyrans to various acid chlorides. The two napthospiropyrans worked with are featured below.

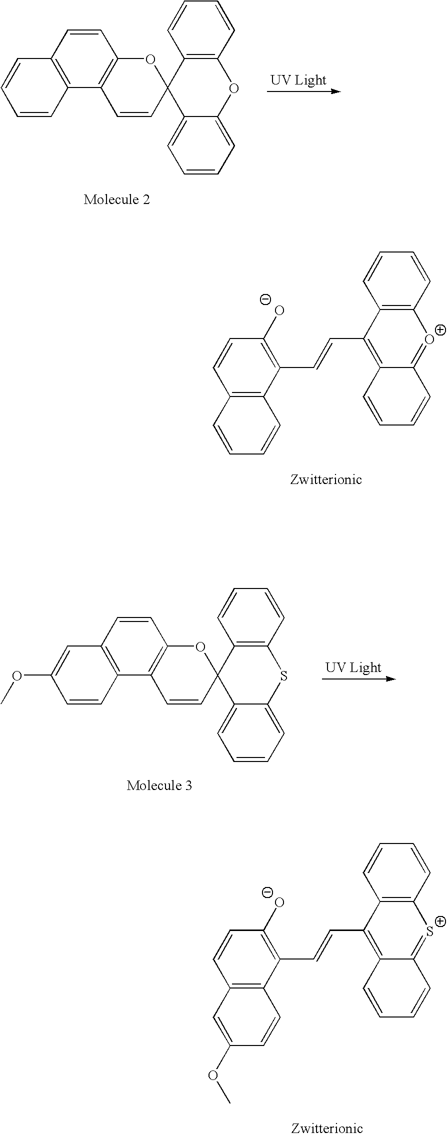

Such napthospiropyrans, are less polarized than spiroindolinopyrans in their open forms, and the issue is whether these would be more susceptible to being trapped out if heteroatoms that could donate charge to the pyran oxygen were placed strategically within the molecule. These napthospiropyrans were tested in hopes of creating a trapped out photochrome that is both resistant to degradation and that exhibits intense color variability.

To start experimentation, two napthospiropyrans were obtained. Molecule 1 is described further below. Molecule 2 was synthesized in 2 steps presented below. Following the completion of this synthesis, the product was purified through column chromatography, which yielded fine white crystals. To obtain Molecule 3, an impure green colored stock of the actual photochrome was purified using column chromatography to obtain a very pure, white crystalline solid. The purity of both of these molecules was further confirmed through H-nmr.

Each of the pure samples was dissolved in dry ether and reacted with acetyl chloride under uv-light. These reactions are presented below.

The following reactions show the steps in synthesizing an oxygen containing napthospiropyran, molecule 2.

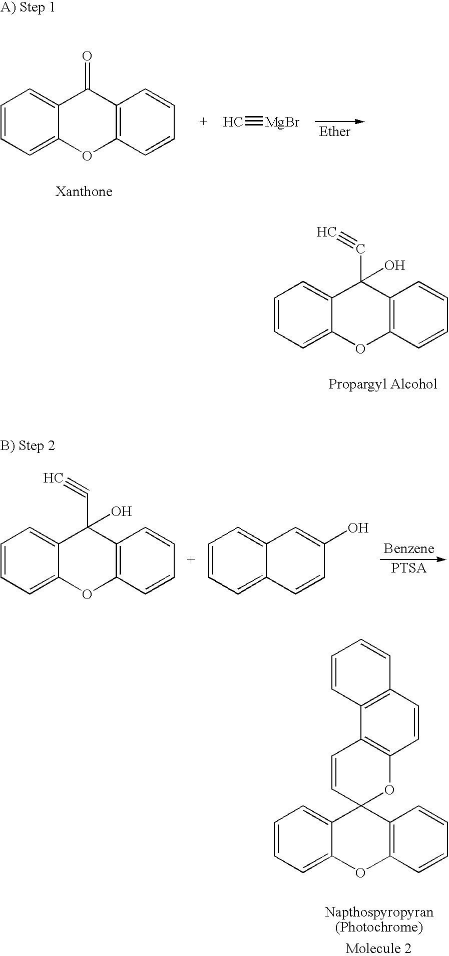

The trapping of Molecule 2 with a proton is as follows.

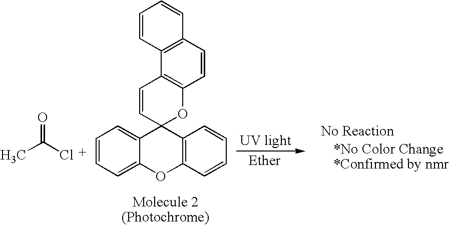

Below is the trapping out of a sulfur containing napthospiropyran, molecule 3, with an acid chloride.

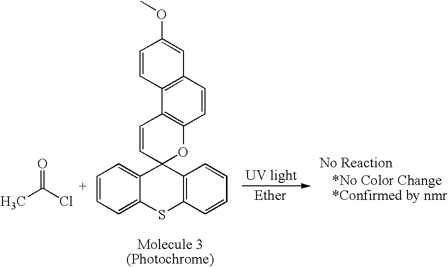

These reaction schemes for molecules 2 and 3 yielded uncolored solutions lacking a solid precipitate. The lack of color change and H-nmr that exhibited only starting products confirmed that the initial reactions were unsuccessful. These results were obtained roughly around the same time that it was discovered that the reaction of Molecule 1 with an acid chloride had not yielded the salt of the ester trapped molecule (see Molecule A below) when the reaction took place in a solution with too much water and/or acid. With respect to Molecule 1, it was believed that a hydrochloric salt of the photochrome was obtained because water present in the ether reacted with the acid chloride to form hydrochloric acid. The hydrochloric acid further reacted with Molecule 1 to create the hydrochloric salt of this photochrome.

In the present circumstances with respect to Molecules 2 and 3, Molecules 2 and 3 were not trapped out successfully with acid chlorides. It was concluded therefore that these molecules did not have enough negative charge on their pyran oxygens in their open forms to act as effective nucleophiles for trapping reactions.

The original reaction of trapping out of Molecule 1 was repeated using anhydrous ether. It also was decided to perform the original reaction of trapping out Molecule 1 with a very electrophilic acid chloride, 2-Benzenesulfonylamino-propionyl chloride, in an extremely dry solvent like anhydrous methylene chloride so as to have a better chance of trapping out Molecule 1 with the desired acid chloride.

The reaction scheme below shows how-acetyl chloride and Molecule 1 (Spiroindolinopyran) in anhydrous ether yielded two products. Molecule A is the photochrome trapped out with acetyl chloride and Molecule B is Molecule 1 trapped out with a proton. Molecule B was formed because the ether used was still wet to some degree.

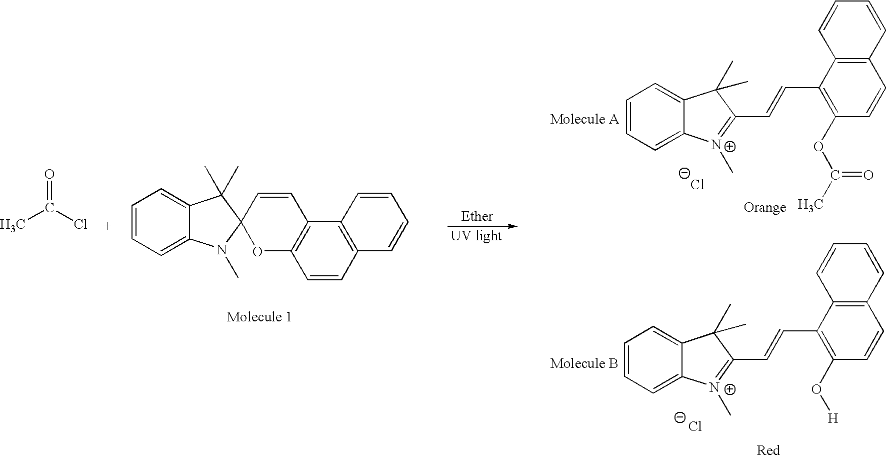

The following scheme illustrates the reaction used to trap out the open form of a spiroindolinopyran with 2-Benzenesulfonylamino-propionyl chloride.

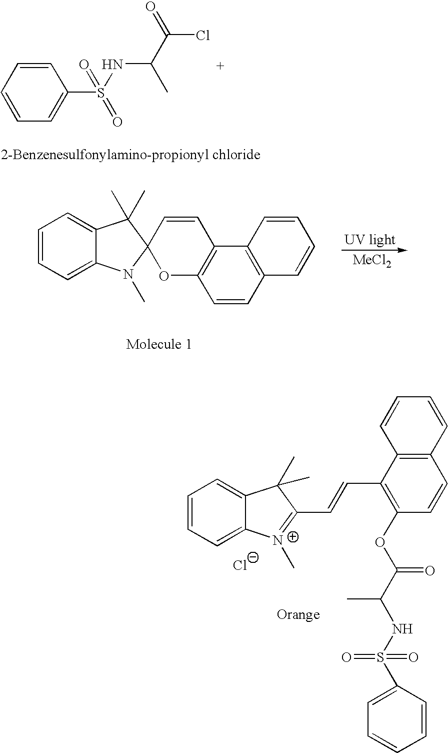

Molecule 1 was successfully trapped out with two different acid chlorides and the acid chloride salts were isolated. Each was more of an orange-like solid when compared to the reddish solid obtained from isolating the hydrochloric salt of Molecule 1. These results were confirmed via H-nmr.

The success in trapping out Molecule 1 with both protons and acid chlorides led to the revival of the original goal to trap out a napthospiropyran with a slight modification. Specifically, trapping would be accomplished with protons. Since structural comparisons of Molecules 2 and 3 indicates that the oxygen containing napthospiropyran would allocate more negative charge to the pyran oxygen than the sulfur containing napthospiropyran, I subjected Molecule 2 to hydrogen chloride gas to trap it out.

The reaction scheme below relates to hydrogen chloride gas being bubbled through an ether solution of Molecule 2.

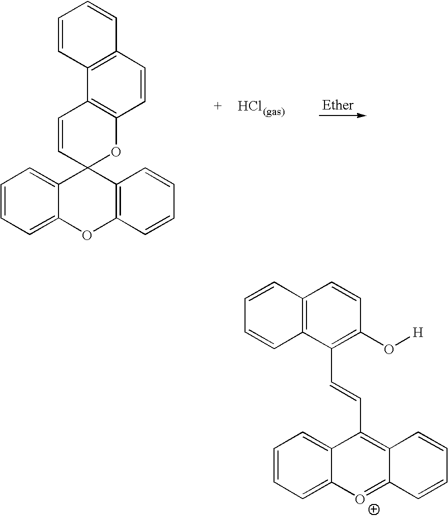

A dark greenish purple solid was obtained, and it is believed that this was our protonated Molecule 2.

Experimental Procedures:

Synthesis of Molecule 2

Two grams (0.0094 moles) of xanthone was placed in a flame dried 500 mL round bottom flask that was purged with nitrogen. 110 mL anhydrous ether was canulated into the flask. 38 mL (0.01884 moles) of ethynyl magnesium bromide was added via syringe under pressure. When the EtMgBr was initially added a whitish precipitate immediately formed which was the product. The reaction was allowed to stir for one week under nitrogen at room temperature.

The product was purified by washing with ether and NaCl₂ saturated water in a separatory funnel. The ether layer was dried with CaCl2 and rotovacced the product to obtain a 1.2 grams of orangeish sticky solid that was the propargyl alcohol.

In a 50 mL round bottom flask, 0.5 grams (0.002248 moles) of the propargyl alcohol, 0.3233 grams of 2-napthol (1 molar equivalent), and 0.05 milligrams of paratoulinic sulfonic acid monohydrate (catalyst) were mixed and refluxed for 4 hours and then purified the final product using glass column chromatography.

Purification of Molecules 2 and 3

For each trial a 4 liter mixture of 95% hexanes and 5% ethyl acetate was made and used as the elution solvent. Silica powder was mixed with the solvent and placed into a glass column. The sample was distributed above the top of the column and sand was placed over the sample. Solvent was then eluded through the column. In both trials white crystalline solids were retained.

Reacting Molecule 2 with Acetyl Chloride

In a 50 mL Erlenmeyer, I dissolved, 28 grams of Molecule 2 in 30 mL of ether. I then added 0.3 mL of acetyl chloride dissolved 5 mL of Ether to the reaction flask. I kept the mixture under nitrogen pressure as I placed the mixture under ultra violet light. The clear colored mixture did not change colors at all and H NMR produced only the unreacted photochrome and acid chloride.

Reacting Molecule 3 with Acetyl Chloride

In a 50 mL Erlenmeyer, I dissolved, 02 grams of Molecule 3 in 30 mL of ether. I then added 0.3 mL of acetyl chloride dissolved 5 mL of Ether to the reaction flask. I kept the mixture under nitrogen pressure as I placed the mixture under ultra violet light. The clear colored mixture did not change colors at all and H NMR produced only the unreacted photochrome and acetyl chloride.

Trapping Molecule 1 with Acetyl Chloride.

A 50 mL round bottom flask was flamed dried and cooled under nitrogen. 0.200 grams of Molecule 1 (6.1×10⁻⁴) was dissolved in 25 mL of anhydrous ether. The solution was slightly pinkish in color. In a separate flame dried 50 mL round bottom, 2 mL of acetyl chloride (roughly 6 molar equivalents) was dissolved in 5 mL of anhydrous ether. The solution was clear.

Each of these round bottoms was sealed with a rubber stopper to prevent water in the air from reacting with the solutions. The dissolved molecule 1 was canulated into the second round bottom containing the acetyl chloride. The mixture was placed under ultra violet light and immediately changed into a bright red color. A nitrogen dried sample of this mixture showed the presence of both the proton and acid chloride trapped photocchrome through H NMR.

Trapping Molecule 1 with 2 Benzenesulfonylamino-Propionyl Chloride.

A 50 mL round bottom flask was flamed dried and cooled under nitrogen. 0.121 grams of Molecule 1 (3.7×10⁻⁴ moles) was dissolved in 25 mL of anhydrous ether. The solution was slightly pinkish in color. In a separate flame dried 50 mL round bottom, 0.097 grams of 2 Benzenesulfonylamino-propionyl chloride (3.7×10⁻⁴ moles) was dissolved in anhydrous ether. The solution was a light tan color.

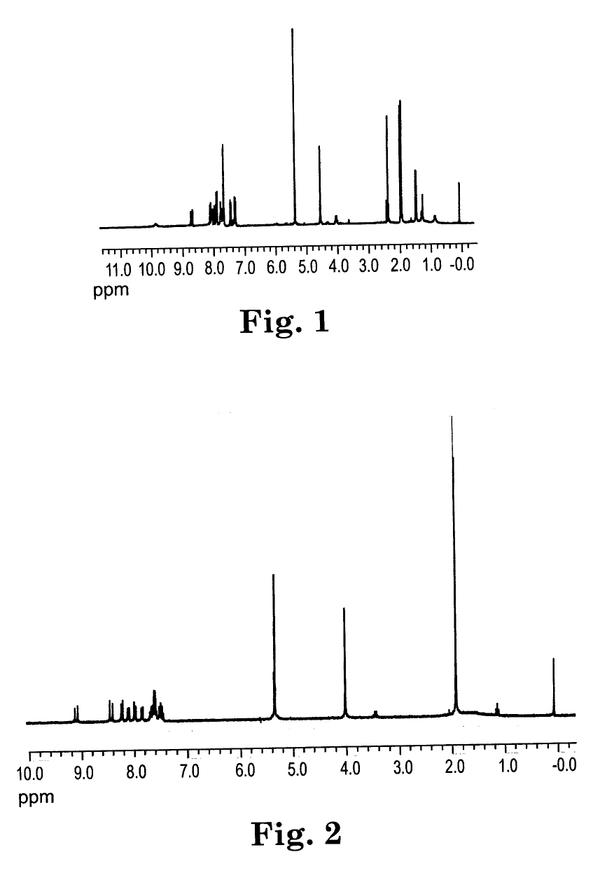

Each of these round bottoms was sealed with a rubber stopper to prevent water in the air from reacting with the solutions. The dissolved molecule 1 was canulated into the second round bottom containing the acid chloride. The mixture was placed under ultra violet light and immediately changed into a bright red color. A nitrogen dried sample of this mixture showed the presence of only the acid chloride trapped photochrome. The characterization of this H-nmr will be completed shortly once more product has been attained. Presented below is the molecule obtained. The corresponding H-nmr obtained is shown in FIG. 1. 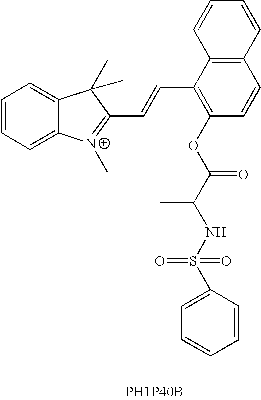
Trapping Molecule 2 with a Proton Using Hydrogen Chloride Gas.

0.0235 grams of Molecule 2 was dissolved in 10 mL of anhydrous ether and capped in a 25mL flame-dried Erlenmeyer flask. Hydrogen chloride gas was bubbled in slowly. The mixture turned a bright turquoise color and then a dark greenish purple solid precipitated.

Results and Discussion of Testing a Thermochromic Compounds as a Chemical Warfare Agent Detection Agent

The novel indicator proposed is a thermochromic compound that exists as an equilibrium mixture of two forms: a closed colorless form and an open colored form. At equilibrium the closed colorless form is highly favored at room temperature and below, whereas the open colored form is only present in a small amount. Through the experiments performed, the attempt was being made to shift the equilibrium of the thermochromic compound by reacting it with diethylchlorophosphate to trap out the open colored state with a structure presumably of that shown below.

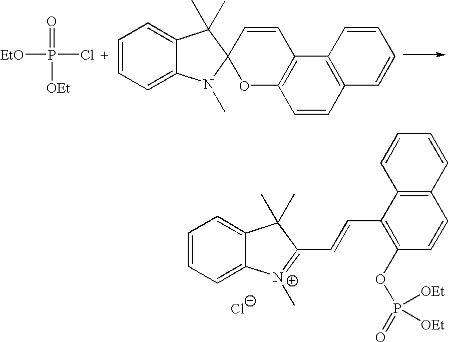

By doing this, the open form could be trapped out causing a visual shift in color. The kinetics were measured to determine a rate constant, and the limits of detection were also determined.

A first experiment was performed to determine if diethylchlorophosphate reacts with the thermochrome the way it is proposed above. The precipitate that crashed out was a red-orange color. The NMR taken is shown in FIG. 2.

The NMR in FIG. 2 showed that diethylchlorophosphate was reacting with the thermochrome in a different way than what was thought.

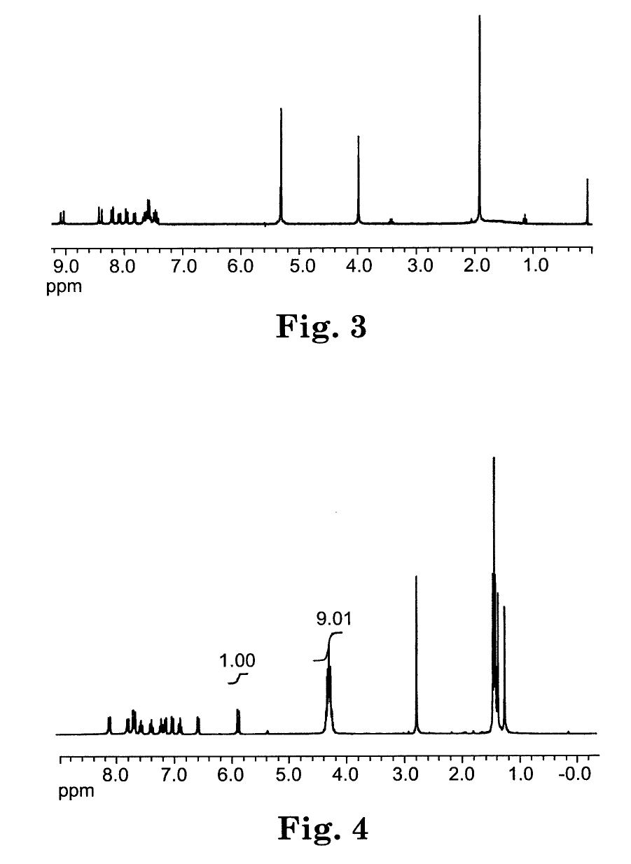

A second experiment was performed to determine if HCl was reacting with the thermochrome and also to isolate the resulting precipitate and characterize it. When the HCl was bubbled into the thermochrome in ether, a red-orange precipitate crashed out. The NMR of this precipitate can be found in FIG. 3. This NMR showed that the water in the ether was actually catalyzing the reaction creating HCl that then reacted with the open form of the thermochrome to create a precipitate. The actual mechanism by which this is postulated to occur is shown below.
Mechanism showing how water catalyzes the reaction between the thermochromic indicator and DCP is as follows:

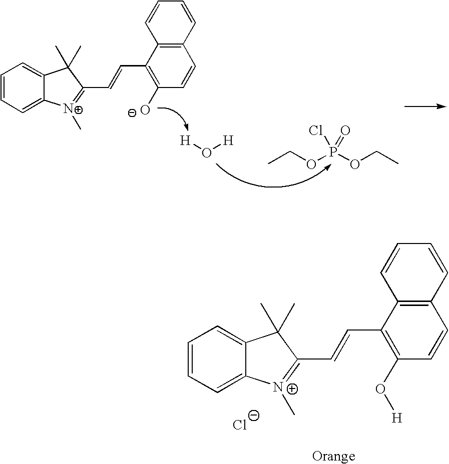

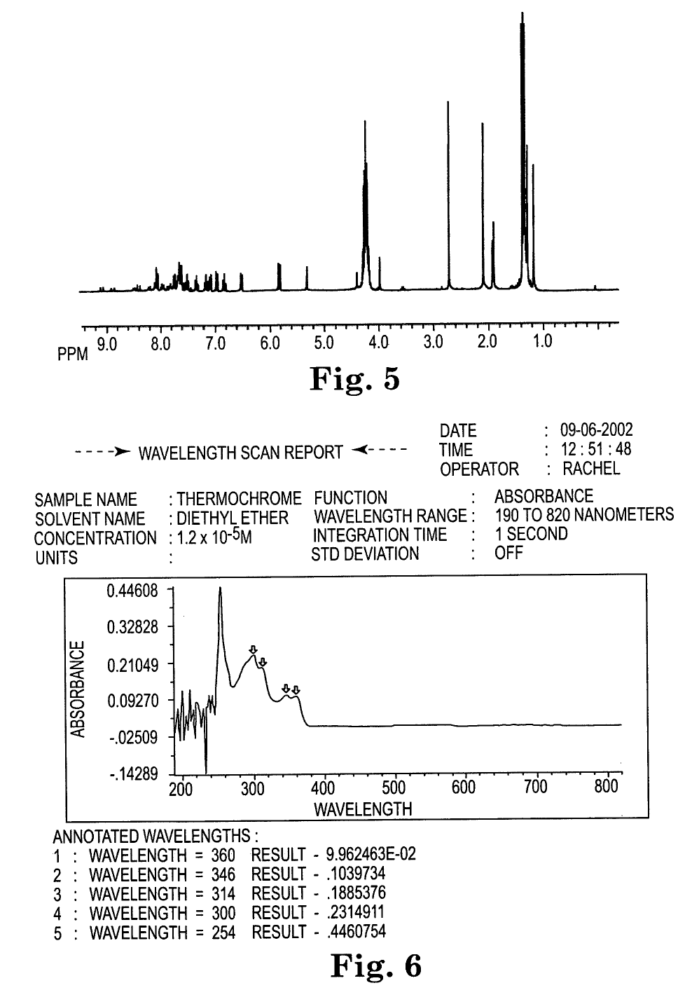

A third experiment was performed to determine if diethylchlorophosphate would react with the thermochrome in the way proposed above with respect to 2-Benzenesulfonylamino-propionyl chloride and molecule 1 in the absence of water. When the deuterated methylene chloride was added to the thermochrome, the solution turned hot pink indicating a shift in equilibrium to give a higher concentration of the open form. When the diethylchlorophosphate was then added to this solution it turned dark red-orange. The first NMR that was taken can be found in FIG. 4. This NMR showed that the starting materials were still present and that they had not reacted significantly yet. The second NMR that was taken a week later can be found in FIG. 5. This NMR showed that the starting materials had started to react. Part of the starting materials had reacted with the diethylchlorophosphate, but some of them had reacted with water to give the protonated form of the thermochrome indicating that water was slowly getting into the NMR tube.

A fourth experiment was performed to determine if diethylchlorophosphate would react with other indicator molecules such as phenolphthalein. When the diethylchlorophosphate was added to the solutions of phenolphthalein (one solution using diethyl ether and one solution using chloroform) there was no observable color change. This showed that other indicator molecules such as phenolphthalein do not react with diethylchlorophosphate as a visual indicator as the novel indicator proposed does.

A fifth experiment was performed to measure the kinetics of the color change when diethylchlorophosphate reacts with the thermochromic compound. The absorption scan of the thermochrome in ether (1.2×10⁻⁵M) showed absorption maximums at 360 nm (9.962463E-02), 346 nm (0.01039734), 314 nm (0.1885376), 300 nm (0.2314911), and 254 nm (0.4460754) (FIG. 6). When the diethylchlorophosphate was mixed with the thermochrome a red-orange color was instantly observed. When the kinetics were measured (on the decay of the wavelength 360 nm over 60 seconds) a flat line was observed. This could have been that the solutions were so dilute that the reaction was taking a long time. To check for this the solutions were made up at a higher concentration and scanned again. This second scan showed a flat line once again. It was determined that the reaction (color change) was happening too fast for the UV-VIS to measure the kinetics of the thermochrome reacting with diethylchlorophosphate. It was determined that an alternative way of measuring the kinetics must be thought of, which is what took place in the second experiment.

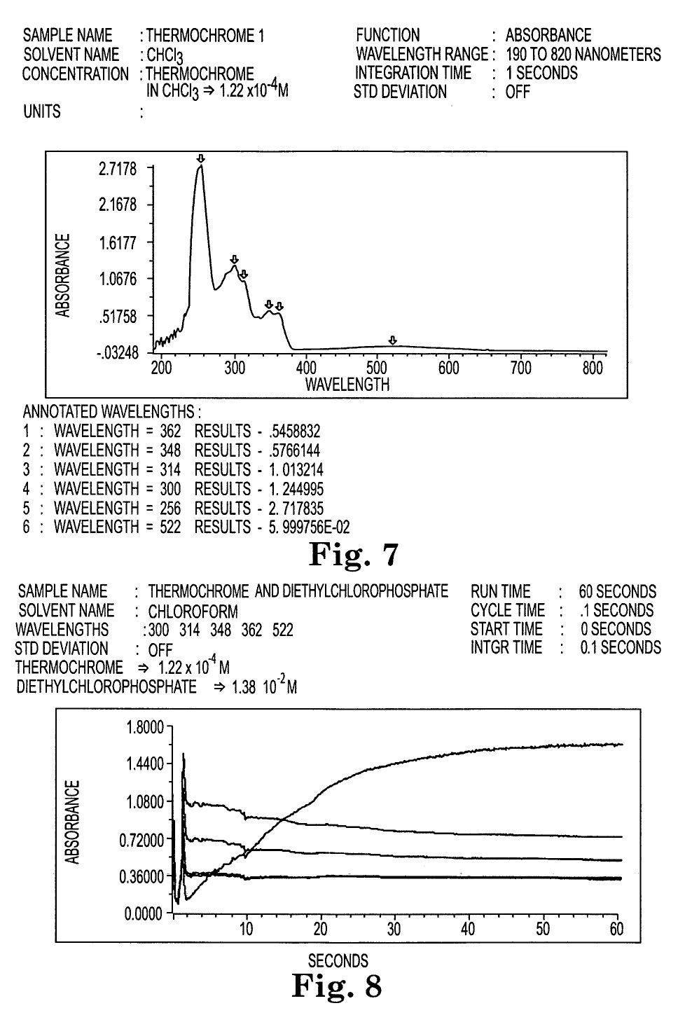

A sixth experiment was performed to measure the kinetics of the color change when diethylchlorophosphate reacts with the thermochromic compound. The absorption scan of the thermochrome in chloroform (1.22×10⁻⁴M) showed absorption maximums at 522 nm (5.999756E-02), 362 nm (0.5458832), 348 nm (0.5766144), 314 nm (1.013214), 300 nm (1.244995), and 256 nm (2.717835) (FIG. 7). When the thermochrome and diethylchlorophosphate solutions were mixed there was an instant color change from a light fuchsia color to hot pink. The absorption scan of the thermochrome (1.22×10⁻⁴M) and diethylchlorophosphate (1.38×10⁻²M) in chloroform over 60 seconds can be found in FIG. 8. The wavelength that was observed at for a rate constant was 522 nm because that was the wavelength in the visible region that represented the visual color change. The five absorbance scans gave the following rate constants in 1/sec with their respective standard deviations: 3.2604E-02+/−2.1920%, 3.6830E-02+/−3.0783%, 3.3525E-02+/−2.4819%, 3.5679E-02+/−2.5497%, 2.7736E-02+/−2.8943%. The average of these five absorbance scans with their standard deviations calculated in gave a rate constant of 3.2406E-02 1/sec to 3.416E-02 1/sec. The consistency throughout the five scans showed that the method was valid and gave reproducible results.

A seventh experiment was performed to determine the limit of detection of the color change when diethylchlorophosphate reacts with the thermochrome. An orange precipitate was observed for each mixture except when the 3.06×10⁻⁴M thermochrome and ether solution was mixed with the 8.625×10⁻⁴M diethylchlorophosphate and ether solution. Because a precipitate could not visually be seen crashing out the experiment was run again with a higher concentrated thermochrome solution. This was to make sure that it was not the concentration of the thermochrome solution that was causing the precipitate to no longer be seen crashing out. This experiment would show that the concentration of diethylchlorophosphate and ether was too dilute to visually detect a precipitate crashing out. When the 3.13×10⁻³M thermochrome and ether solution was mixed with the 8.625×10⁻⁴M diethylchlorophosphate and ether solution a precipitate was not observed. This showed that the lowest visual color change that could be seen occurs when the molarity of the diethylchlorophosphate and ether solution is greater than 8.625×10⁻⁴M but less then 1.725×10⁻³M.

These experiments showed that the novel indicator proposed reacts with diethylchlorophosphate in a different way then first thought. Although it reacts differently it is still effective as a visual indicator for nerve gas. The color change that occurs has a measured rate constant of 3.2406E-02 1/sec to 3.416E-02 1/sec. The lowest visual color change that the indicator produces when reacting with diethylchlorophosphate in ether is found at a molarity that is greater than 8.625×10⁻⁴M but less then 1.725×10⁻³M.

Experimental Procedure for Chemical Warfare Agent Detection Results and Discussion discussed directly above.

### EXPERIMENTAL

The thermochromic indicator used throughout every experiment below has the structure of molecule 1, described above. The diethylchlorophosphate was from Aldrich, Batch number 12616AB. The diethyl ether used was from Spectrum, Lot number RG0250. The chloroform used was from Mallinckrodt, Lot number 4440KTEP. The deuterated methylene chloride used was from Cambridge Isotope Laboratories. The HCl gas used was from Aldrich, Lot number 04548BO. The phenolphthalein was from Mallinckrodt, Product number 6600. The ultra-violet visible absorbance spectrophotometer (UV-VIS) used was the Hewlett Packard 8452A Diode Array Spectrophotometer. The nuclear magnetic resonance (NMR) machine used was the Bruker ACP-300.

### Experiment 1

Reaction Between Diethylchlorophosphate and the Thermochrome in Ether

100 mg of the thermochrome was weighed out in a 125 mL Erlenmeyer flask. 25 mL of the diethyl ether was then added to dissolve the thermochrome. This Erlenmeyer flask was then capped and degassed with nitrogen. 4.42 mL of diethylchlorophosphate was measured out in the glove box and put in a 10 mL Erlenmeyer flask and capped. It was then taken out of the glove box. In the hood the 4.42 mL of diethylchlorophosphate was taken out of the 10 mL Erlenmeyer flask with a syringe and put into the flask containing the thermochrome and diethyl ether solution. The precipitate that crashed out was filtered with a size D glass frit filter. An NMR was taken of the precipitate in deuterated methylene chloride.

### Experiment 2

Reaction Between HCl and the Thermochrome

0.001 g of the thermochrome was weighed out in a 25 mL Erlenmeyer flask. 10 mL of diethyl ether was then added to the 25 mL Erlenmeyer to dissolve the thermochrome. The solution was then capped. A tank of HCl gas was then set up with a line from the tank going into the thermochrome solution and a needle in the cap of the thermochrome solution to vent it. HCl was then bubbled through the thermochrome solution. The precipitate formed was filtered using a size D glass frit filter. An NMR was taken of the precipitate in methylene chloride. This experiment was repeated using chloroform instead of diethyl ether. Instead of a precipitate being filtered and analyzed through a NMR, the color change was noted.

### Experiment 3

Reaction Between Diethylchlorophosphate and the Thermochrome

A pre-weighed sample of the thermochromic compound, diethylchlorophosphate, deuterated methylene chloride, a vial, pipettes, spatula, microliter syringe, and an NMR tube were all put in the glove box. The glove box was flushed with nitrogen gas three times. The rest of the experiment (with the exception of the NMR) was done in the glove box under nitrogen. 0.0100 g of the thermochrome was weighed into a vial. 0.5 mL of deuterated methylene chloride was added to this vial to dissolve the thermochrome. 4.42 microliters of diethylchlorophosphate was then measured out with a syringe. This was then added to the vial containing the thermochrome in deuterated methylene chloride. This solution was then put into a NMR tube and capped. The NMR tube was taken out of the glove box and an NMR was taken. The NMR tube was put in the fridge. After a week had gone by the NMR tube was taken out of the fridge and another NMR was taken.

### Experiment 4

Reaction Between Phenolphthalein and Diethylchlorophosphate

10 mg of phenolphthalein was weighed out in a 25 mL Erlenmeyer flask. 10 mL of diethyl ether was added to dissolve the phenolphthalein. 4.54 microliters of diethylchlorophosphate was added to the phenolphthalein and diethyl ether solution. Whether or not there was a color change was noted. This experiment was repeated except chloroform was used instead of diethyl ether.

### Experiment 5

Measure of Kinetics

A sample of diethyl ether was put in a quartz cuvette and scanned as a blank on the ultra-violet visible absorbance spectrophotometer (UV-VIS). A solution of the thermochromic compound dissolved in diethyl ether was made up at a molarity of 1.2×10⁻⁵. The same cuvette used above was emptied out and dried with nitrogen gas. A sample of the 1.2×10⁻⁵M thermochrome and diethyl ether solution was then put in the cuvette and its absorbance was scanned using the UV-VIS. A solution of diethylchlorophosphate and diethyl ether was made up at a molarity of 1.2×10⁻³. The UV-VIS was set to scan the kinetics at 360 nm. 3 drops of the 1.2×10⁻³M diethylchlorophosphate and diethyl ether solution was added to the sample of 1.2×10⁻⁵M thermochrome and diethyl ether solution in the cuvette. This was quickly stirred and then the kinetics were measured using the UV-VIS to see the decay of the wavelength 360 nm over 60 seconds. This experiment was repeated using a 3.058×10⁻³M solution of the thermochrome and diethyl ether instead of the 1.2×10⁻⁵M solution and a 1.384×10⁻²M solution of the diethylchlorophosphate and diethyl ether instead of the 1.2×10⁻³M solution.

### Experiment 6

Second Measure of Kinetics

A sample of chloroform was put in a quartz cuvette and scanned as a blank on the UV-VIS. A solution of the thermochromic compound dissolved in chloroform was made up at a molarity of 1.22×10⁴. The same cuvette used above was emptied out and dried with nitrogen gas. A sample of the 1.22×10⁴M thermochrome and chloroform solution was then put in the cuvette and its absorbance was scanned using the UV-VIS. A solution of diethylchlorophosphate and chloroform was made up at a molarity of 1.38×10⁻². The cuvette used above was then emptied out, dried with nitrogen gas, and placed back in the UV-VIS empty. A 3 mL syringe was filled to the 1.5 mL line with the 1.22×10⁴M thermochrome and chloroform solution. Another 3 mL syringe was filled to the 1.5 mL line with the 1.38×10⁻²M diethylchlorophosphate and chloroform solution. The UV-VIS was set to take the kinetics (to determine a rate constant) of the following wavelengths: 300, 314, 348, 362, and 522 nm. On the kinetics module the scan was set to take place over 60 seconds. The cycle was set to take a scan every 0.1 seconds with the integer also set at 0.1 seconds. A syringe was put in each hand. An assistant helped with this part of the experiment by pressing the button to start the scan at the same time that I squirted the thermochrome solution and the diethylchlorophosphate solution from the syringes into the empty cuvette. The results from this UV-VIS scan were then used to calculate a rate constant for each wavelength. This procedure was repeated four more times to show reproducibility and validity.

### Experiment 7

Limits of Detection

A solution of the thermochrome and diethyl ether was made up at a molarity of 3.06×10⁴. A solution of diethylchlorophosphate and diethyl ether was made up at a molarity of 1.384×10⁻². A 5 mL sample of the 3.06×10⁴M thermochrome solution and a 5 mL sample of the 1.384×10⁻²M diethylchlorophosphate solution were mixed in a 25 mL Erlenmeyer flask and whether or not a precipitate crashed out was noted. The diethylchlorophosphate and diethyl ether solution was diluted to a molarity of 6.9×10⁻³. A 5 mL sample of the 3.06×10⁻⁴M thermochrome solution and a 5 mL sample of the 6.9×10⁻³M diethylchlorophosphate solution were mixed in a 25 mL Erlenmeyer flask and whether or not a precipitate crashed out was noted. The diethylchlorophosphate and diethyl ether solution was diluted to a molarity of 3.45×10⁻³. A 5 mL sample of the 3.06×10⁻⁴M thermochrome solution and a 5 mL sample of the 3.45×10⁻³M diethylchlorophosphate solution were mixed in a 25 mL Erlenmeyer flask and whether or not a precipitate crashed out was noted. The diethylchlorophosphate and diethyl ether solution was diluted to a molarity of 1.73×10⁻³. A 5 mL sample of the 3.06×10⁻⁴M thermochrome solution and a 5 mL sample of the 1.73×10⁻³M diethylchlorophosphate solution were mixed in a 25 mL Erlenmeyer flask and whether or not a precipitate crashed out was noted. The diethylchlorophosphate and diethyl ether solution was diluted to a molarity of 8.63×10⁻⁴. A 5 mL sample of the 3.06×10⁻⁴M thermochrome solution and a 5 mL sample of the 8.63×10⁻⁴M diethylchlorophosphate solution were mixed in a 25 mL Erlenmeyer flask and whether or not a precipitate crashed out was noted. A solution of the thermochrome and diethyl ether was made up at a molarity of 3.13×10⁻³. A 5 mL sample of the 3.13×10⁻³M thermochrome solution and a 5 mL sample of the 8.63×10⁻⁴M diethylchlorophosphate solution were mixed in a 25 mL Erlenmeyer flask and whether or not a precipitate crashed out was noted.

1. A method of making a trapped chromogenic compound comprising the step of causing an open form of a chromogenic compound and a compound comprising an electrophilic moiety to be covalently bonded.

2. The method of claim 1, wherein the chromogenic compound comprises a photochromic compound or a thermochromic compound.

3. The method of claim 1, wherein the compound comprising an electrophilic moiety comprises an acyl functional compound.

4. The method of claim 1, wherein the compound comprising an electrophilic moiety comprises an enzyme substrate.

5. A trapped, open, chromogenic compound useful for detecting an enzyme made using the procedure of claim 4.

6. A trapped, open, chromogenic compound made by the method of claim 1.

7. The trapped, open, chromogenic compound of claim 6, comprising an ester moiety derived from ingredients comprising an oxygen atom of an open pyran moiety and an acyl functional compound.

8. A method of untrapping the trapped, open, chromogenic compound of claim 6 comprising the step of cleaving the chromogenic compound via a hydrolysis reaction mechanism to provide the closed form of the chromogenic compound.

9. The method of claim 8, wherein the chromogenic compound comprises an ester moiety derived from ingredients comprising an oxygen atom of an open pyran moiety and an acyl functional compound and wherein the acyl functionally compound is cleaved via the hydrolysis reaction mechanism.

10. A process for detecting the presence of a biological molecule comprising the step of untrapping the trapped, open, chromogenic compound according to the method of claim 8, wherein the hydrolysis reaction is indicative of the presence of the biological molecule.

11. The process of claim 10, wherein the biological molecule is selected from the group consisting of an enzyme, protein, RNA, DNA, and sugar.

12. A process for detecting the presence of a toxic chemical comprising the step of untrapping the trapped, open, chromogenic compound according to the method of claim 8, wherein the hydrolysis reaction is indicative of the presence of the toxic chemical.

13. The process of claim 12, wherein the toxic chemical comprises nerve agent.

14. A photochromic compound trapped in an open configuration and having the following structure:
Z-W—R
wherein Z is derived from ingredients comprising any photochomic compound including a pyran moiety and Z is present in a configuration in which the pyran moiety is open, W is a single bond or a divalent linking group linked to the open pyran moiety, and R is at least a portion of the residue of a compound comprising an electrophilic moiety.

15. The photochromic compound of claim 14, wherein Z is derived from ingredients comprising an indolinospiropryan.

16. The photochromic compound of claim 14, wherein R is cleavable in the presence of an enzyme.

17. The photochromic compound of claim 14, wherein W comprises an ester moiety.

18. The photochromic compound of claim 14, wherein the compound is obtained by trapping out Photochrome I with an electrophile Q according to the reaction scheme shown below or wherein the Photochrome II is trapped out with the electrophile Q using the same reaction scheme: 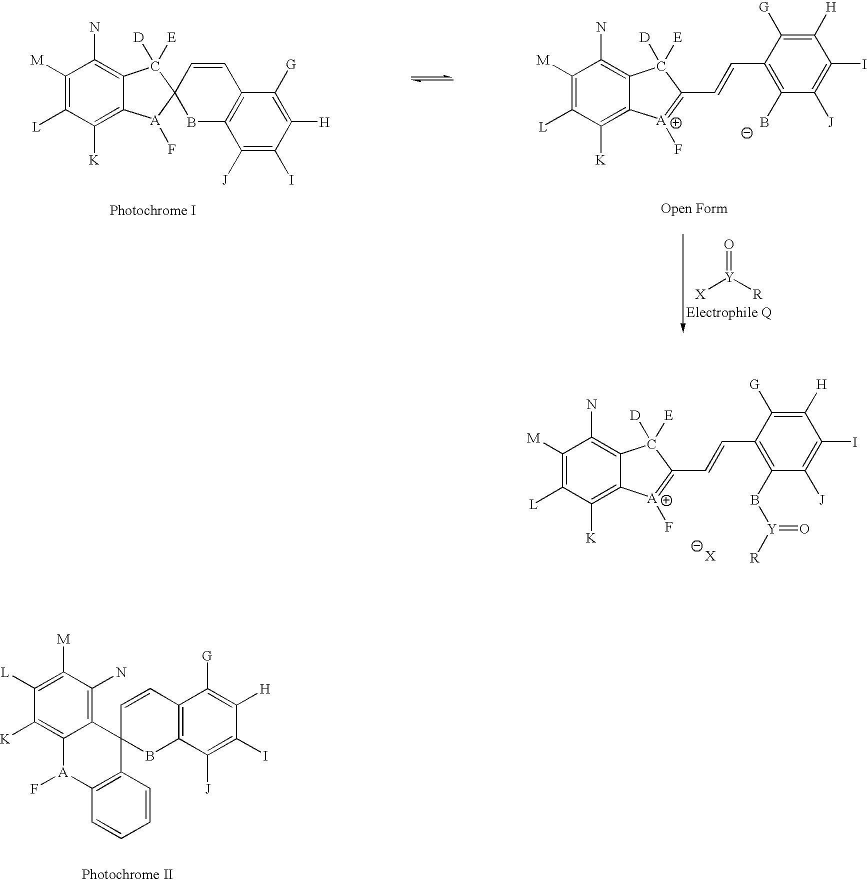
wherein the substituent R on the electrophile Q is any monovalent moiety; X is an anion; B is a connective moiety; A comprises N or C; and each of the D, E, F, G, H, I, J, K, L, M, N substituents independently comprise any monovalent moiety or a member of a cyclic substituent; the substituent C is selected from the group consisting of carbon, sulfur, nitrogen, and another heteroatom.

19. The photochromic compound of claim 18, wherein Q comprises an enzyme substrate.

20. The photochromic compound of claim 18, wherein X comprises a halide.

21. The photochromic compound of claim 18, wherein B is selected from the group consisting of oxygen, sulfur, and nitrogen.

22. The photochromic compound of claim 18, wherein the substituent C comprises C(R″), wherein R″ comprises a monovalent moiety selected from the group consisting of hydrogen, and an alkyl moiety.

23. The photochromic compound of claim 18, wherein A comprises nitrogen, B comprises oxygen, C comprises carbon, D, E, and F comprise methyl, G and H are co-members of an aromatic ring structure, Y comprises carbon, R comprises an enzyme substrate, and I, J, K, L, M, and N are each independently selected from the group consisting of any monovalent substituent, alkyl, co-member of a ring structure, aromatic structure, halide, sulfonate, phosphonate, NO₂, alkoxy, and carboxyl.

24. The photochromic compound of claim 18, wherein C comprises sulfur and F comprises phenyl.

25. A method for directly or indirectly detecting the presence or activity of an enzyme in a sample comprising:
(a) contacting the sample with a photochromic compound trapped in an open configuration and having a structure according to claim 14 wherein R comprises a moiety reactive with the enzyme; and
(b) visually observing the photochromic compound for any change in color, wherein any such color change indicates the presence of the enzyme.

26. A method for detecting the presence of a toxic chemical in a sample comprising:
(a) contacting the sample with a photochromic compound capable of being trapped into an open configuration via covalent bonding with the toxic chemical; and
(b) visually observing the photochromic compound for a change in color.

27. The method of claim 26, wherein the toxic chemical comprises a nerve agent.

28. A method of providing a detection molecule comprising the steps of determining information indicative of the rate at which an untrapped, open chromophore reverts to a closed configuration; and using the information to make a trapped open chromophore having a desired calorimetric rate of response in a detecting application.
